(19) United States
(12) Patent Application Publication
(10) Pub. No.: US 2012/0178099 A1
(43) Pub. Date: Jul. 12, 2012

(54) HIGHLY FLUORESCENT CARBON NANOPARTICLES AND METHODS OF PREPARING THE SAME

(75) Inventor: Nikhil Ranjan JANA, KOLKATA (IN)

(73) Assignee: INDIAN ASSOCIATION FOR THE CULTIVATION OF SCIENCE, Kolkata (IN)

(21) Appl. No.: 13/346,401

(22) Filed: Jan. 9, 2012

(30) Foreign Application Priority Data
Jan. 10, 2011 (IN) 22/KOL/2011

(51) Int. Cl. G01N 21/64; B32B 5/16; C01B 31/02; C12Q 1/02; B82Y 15/00; B82Y 40/00

(52) U.S. Cl. 435/7.1; 423/445 R; 435/29; 428/402; 977/896; 977/773; 977/906

(57) ABSTRACT

Highly fluorescent carbon nanoparticles (FCNs), with tunable emission colours of particle size between 1-10 nm also stable in solid form with high quantum yield (>5%) and its method of synthesis thereof yielding said carbon nanoparticles in milligram to gram scale in high synthesis yield (>80%). The present invention also provides for highly fluorescent carbon nanoparticle solution doped with heteroatom (such as oxygen, nitrogen) and its method of synthesis favoring yield of the said doped carbon nanoparticles of even smaller size ranging from 1-5 nm with narrow size distribution, and also provides for functionalized FCNs that are non-toxic, functional, soluble and stable fluorescent carbon nanoparticles with retained fluorescence for variety of end uses in biomedics, imaging applications, and detection techniques.

### CROSS-REFERENCE TO RELATED APPLICATION AND CLAIM OF PRIORITY

This application claims the benefit of Indian Patent Application No. 22/KOL/2011, filed on Jan. 10, 2011, in the Indian Patent and Trademark Office, the disclosures of which are incorporated herein in their entirety by reference.

### BACKGROUND OF THE INVENTION

1. Field of the Invention

The present invention relates to highly fluorescent carbon nanoparticles (FCN), with tunable emission colours of particle size between 1-10 nm with quantum yield (>5%) that is also stable in solid form, and also, relates to its method of synthesis. Advantageously, the present invention provides for yield of the said carbon nanoparticles in milligram to gram scale in high synthesis yields (>80%). Further, the present invention deals with highly fluorescent carbon nanoparticle solution doped with heteroatom (such as oxygen, nitrogen) and its method of synthesis favoring yield of the said doped carbon nanoparticles of even smaller size ranging from 1-5 nm with narrow size distribution. More advantageously, the present invention also relates to the functionalization of the said FCNs to provide for non-toxic, functional, soluble and stable fluorescent carbon nanoparticles with retained fluorescence and its method of synthesis for variety of end uses in biomedics, imaging applications, and detection techniques.

2. Description of the Related Art

Fluorescent carbon nanoparticles (FCN) are increasingly getting more attention in recent years due to their tunable fluorescence in visible region and reported synthetic methods with high quantum yield. They have high potential in biological labeling due to lower toxicity [Li, Q., Ohulchanskyy. T. Y., Liu, R., Koynov, K., Wu, D., Best, A., Kumar, R., Bonoiu, A. & Prasad, P. N. Photoluminescent Carbon Dots as Biocompatible Nanoprobes for Targeting Cancer Cells in Vitro. J. Phys. Chem. C 114, 12062-12068 (2010)] compared to conventional cadmium based quantum dots [Medintz, I. L., Uyeda, H. T., Goldman, E. R. & Mattoussi, H. Quantum dot bioconjugates for imaging, labelling and sensing. Nat. Mater. 4, 435-446 (2005), Pelley, J. L., Daar, A. S., Saner, M. A. State of Academic Knowledge on Toxicity and Biological Fate of Quantum Dots. Toxicological Sciences 112, 276-296 (2009)]. However, they are little studied compared to other carbon based materials such as fullerene [Diederich, F. & Thilgen, C. Covalent fullerene chemistry. Science 271, 317-323 (1996)], carbon nanotube [Tasis, D., Tagmatarchis, N., Bianco, A. & Prato, M. Chemistry of carbon nanotubes. Chem. Rev. 106, 1105-1136 (2006)] and graphene [Dreyer, D. R., Park, S., Bielawski, C. W. & Ruoff, R. S. The chemistry of graphene oxide. Chem. Soc. Rev. 39 (228-240) 2010]. Currently fluorescent carbon nanoparticles are synthesized via both physical and chemical approaches. Physical methods include high energy radiation based creation of point defect in diamond particle [Gruber, A., Drabenstedt A., Tietz, C., Fleury, L., Wrachtrup, J., vonBorczyskowski, C. Scanning confocal optical microscopy and magnetic resonance on single defect centers. Science, 276, 2012-2014 (1997), Yu, S. J., Kang, M. W., Chang, H. C., Chen, K. M. & Yu, Y. C. Bright fluorescent nanodiamonds: No photobleaching and low cytotoxicity. J. Am. Chem. Soc. 127, 17604-17605 (2005)]. and laser ablation of graphite [Sun, Y. P., Zhou, B., Lin, Y., Wang, W., Fernando, K. A. S., Pathak, P., Meziani, M. J., Harruff, B. A., Wang, X., Wang, H. F., Luo, P. J. G., Yang, H., Kose, M. E., Chen, B. L., Veca, L. M. & Xie, S. Y. Quantum-sized carbon dots for bright and colorful photoluminescence. J. Am. Chem. Soc. 128, 7756-7757 (2006)]. Chemical methods include oxidation of candle soot, [Liu, H., Ye, T. & Mao, C. Fluorescent carbon nanoparticles derived from candle soot. Angew. Chem. Int. Ed. 46, 6473-6475 (2007)], Ray, S. C., Saha, A., Jana, N. R. & Sarkar, R. Fluorescent Carbon Nanoparticles: Synthesis, Characterization, and Bioimaging Application. J. Phys. Chem. C 113, 18546-18551 (2009)] carbonization of carbohydrate[Selvi, B. R., Jagadeesan, D., Suma, B. S., Nagashankar, G., Arif, M., Balasubramanyam, K., Eswaramoorthy, M. & Kundu, T. K. Intrinsically Fluorescent Carbon Nanospheres as a Nuclear Targeting Vector: Delivery of Membrane-Impermeable Molecule to Modulate Gene Expression In Vivo. Nano Lett. 8, 3182-3188 (2008), Peng, H., Travas-Sejdic, J. Simple aqueous solution route to luminescent carbogenic dots from carbohydrates. Chem. Mater. 21, 5563-5565 (2009), Zhang, J., Shen, W., Pan, D., Zhang, Z., Fang, Y. & Wu, M. Controlled synthesis of green and blue luminescent carbon nanoparticles with high yields by the carbonization of sucrose. New J. Chem. 34, 591-593 (2010)], thermal decomposition of citrate [Bourlinos, A. B., Stassinopoulos, A., Anglos, D., Zboril, R., Georgakilas, V. & Giannelis, E. P. Photoluminescent carbogenic dots. Chem. Mater. 20, 4539-4541 (2008), Bourlinos, A. B., Stassinopoulos, A., Anglos, D., Zboril, R., Karakassides, M., & Giannelis, E. P. Surface functionalized carbogenic quantum dots. Small, 4, 455-458 (2008), Wang, F., Pang, S., Wang, L., Li, Q., Kreiter, M. & Liu, C.-Y. One-Step Synthesis of Highly Luminescent Carbon Dots in Noncoordinating Solvents. Chem. Mater. 22, 4528-4530 (2010)], pyrolysis of ethylenediaminetetraacetic acid [Pan, D., Zhang, J., Li, Z., Wu, C., Yan, X., Wu, M. Observation of pH-, solvent-, spin-, and excitation-dependent blue photoluminescence from carbon nanoparticles. Chem. Commun. 46, 3681-3683 (2010)], degradation of ascorbate [Zhang, B., Chun-yan Liu, C.-Y. & Liu, Y. A Novel One-Step Approach to Synthesize Fluorescent Carbon Nanoparticles. European Journal of Inorganic Chemistry 26 AUG 2010, DOI: 10.1002/ejic.201000622] and pyrolysis of phenol-formaldehyde resin [Liu, R., Wu, D., Liu, S., Koynov, K., Knoll, W. & Li, Q. An Aqueous Route to Multicolor Photoluminescent Carbon Dots Using Silica Spheres as Carriers. Angew. Chem. Int. Ed. 48, 4598-4601 (2009)]. Improvement of emission quantum yield, tunablity of emission colour, understanding of the origin of fluorescence, surface functionalization and testing of in vitro and in vivo imaging are the critical issues that need to solved prior to their wider application [Baker, S. N. & Baker, G. A. Luminescent Carbon Nanodots: Emergent Nanolights.

Angew. Chem. Int. Ed. 49, 6726-6744 (2010)].

There are two distinct limitations in the preparation and application of FCN [Baker, S. N. & Baker, G. A. Luminescent Carbon Nanodots: Emergent Nanolights.

Angew. Chem. Int. Ed. 49, 6726-6744 (2010), Wang, X., Cao, L., Yang, S.-T., Lu, F., Meziani, M. J., Tian, L., Sun, K. W., Bloodgood, M. A. & Sun, Y.-P. Bandgap-Like Strong Fluorescence in Functionalized Carbon Nanoparticles. Angew. Chem. Int. Ed. 49, 5310-5314 (2010), Li, Q., Ohulchanskyy. T. Y., Liu, R., Koynov, K., Wu, D., Best, A., Kumar, R., Bonoiu, A. & Prasad, P. N. Photoluminescent Carbon Dots as Biocompatible Nanoprobes for Targeting Cancer Cells in Vitro. J. Phys. Chem. C 114, 12062-12068 (2010)]. First, no methods are available for large scale synthesis of FCN. Particularly FCN with high fluorescence quantum yield are produced in very low yield and preparation of milligram scale of such FCN is a difficult task. Second, most synthetic methods produce weakly fluorescent FCN with <1% quantum yield.

Reference is drawn to patent publication US 20080113448, published on May 15, 2008 which discloses photo-luminescent particles having a core nano-sized particle of carbon and a passivation agent bound to the surface of the nanoparticles and a complex method of specialized laser ablation of graphite and electric arc discharge of carbon powder. Additional complex surface passivation and oxidation via multiple steps are necessary to exhibit the fluorescence. The produced particles are polydispersed in nature and size ranges between 1-100 nm. Thus particles with mixture of different emission colors are present.

An article published in Journal of Physical Chemistry, 113, 18546, 2009 discloses a method for preparing carbon particles with a maximum of 3% quantum yield. The method needs significant effort to prepare 2-3 mg of such sample. The need of increasing the quantum yield and large scale preparation is a challenge to the present day researchers.

Yet another reference is drawn to US20100215760, published on August 2010 that teaches a method for carbonization of carbohydrate for large scale synthesis; however, quantum yield is very low (<1%) and produces large size particles with only blue and green emissions The produced particles are either larger in size (>10 nm) and non-fluorescent or weakly fluorescent with quantum yield 0.01-0.13%. Although carbohydrate molecules have been used for the synthesis of carbon nanoparticles, the said method did not lead to any favourable results.

Further references may be made to articles from Chemistry of Materials, 20, 4539, 2008 by A. B. Bourlinos et al., Angew. Chem. Int. Ed. 48, 4598, 2009 by R. Liu et al and Chemical Communication, 46, 3681, 2010 by D. Pan et al, which disclose carbonization of citrate, polymer, EDTA, to produce intense blue emission; however suffer from limitations like difficulty of tunability of emission colors and low quantum yield for other colors.

As apparent from the abovesaid, till date the prior methods lack in the large scale synthesis of FCN with high fluorescence quantum yield. Teachings flowing from the background art reveal that FCNs are commonly produced in very low yield and preparation of milligram scale of such FCN is a difficult task. Most synthetic methods produce weakly fluorescent FCN with <1% quantum yield. Though in some prior art methods the quantum yields vary between 5 to 60%, such methods involve sophisticated high energy radiation followed by surface functionalization with certain molecules and gel based size separation which are cost expensive and enables attaining less than milligram scale of FCNs and hence rendering the entire process difficult and non-economical. Moreover, most synthetic methods reported in the art produces FCN with one emission color and some methods produce mixture of FCN with different emission colors that requires specialized gel based size separation methods to isolate FCN of different emission colors. Further to this, functionalization of FCN is relatively unexplored area with limited success. As apparent from the aforesaid it is thus desirable to produce highly fluorescent FCNs in a large scale (>milligram scale) with >5% quantum yield and with tunable emission colors.

Therefore there is a longfelt need in the art to provide for highly fluorescent carbon nanoparticles solids/aqueous and non-aqueous solutions with tunable emission colours of reduced particle size and its method of synthesis thereof to yield the said carbon nanoparticles in milligram to gram scale in high quantum yield and high synthetic yield. Also there is a strong need in the art to suitably functionalize the FCNs, to overcome the drawbacks of the prior art, and to find its end use and application in biomedics and various cellular, sub-cellular, in-vivo and in-vitro imaging applications and detection techniques.

### SUMMARY

The primary object of the present invention is thus directed to provide for a highly fluorescent carbon nanoparticles involving aqueous and non-aqueous solutions of hydrophobic/hydrophilic fluorescent carbon nanoparticles with tunable emission colours and reduced particle size of 1-10 nm that would be also stable in solid form.

Another object of the present invention is to provide for said fluorescent carbon nanoparticles which are stable in air and under light irradiation both in solution and in solid form.

Yet another object of the present invention is to provide for water soluble and functional fluorescent carbon nanoparticles with good colloidal stability in a medium with pH range between 4 and 10 without any loss of fluorescence property.

Yet another object of the present invention is to provide for a simple and cost effective method of synthesis to yield said highly fluorescent carbon nanoparticle solution in milligram to gram scale in high quantum yield of >5% and high synthetic yield of >80% that would emit different color emission spectra such as blue, green, yellow and red in visible to NIR regions.

Another object of the present invention is to provide for a highly fluorescent carbon nanoparticle solution doped with heteroatom (such as oxygen, nitrogen) and its method of synthesis to yield said doped carbon nanoparticles of even smaller size with narrow size distribution.

Still another object of the present invention is to provide for non-toxic, functional, soluble and stable fluorescent carbon nanoparticles with retained fluorescence and its method of synthesis for its end use in biomedics, various imaging applications, and detection techniques.

Thus according to the basic aspect of the present invention there is provided Fluorescent carbon nanoparticles (FCNs) comprising carbon matrix based nanoparticles of upto 10 nm in size with variably tunable emission colours and being highly fluorescent with quantum yield of >5%.

In another preferred aspect of the present invention there is provided said fluorescent carbon nanoparticles (FCNs) having highly fluorescent quantum yield in the range of 5-15% and tunability of emission colours of blue, green, yellow, red with emission peaks at 455 nm (excitation at 350 nm), 480 nm (excitation at 400 nm), 520 nm (excitation at 400 nm), 540 nm (excitation at 450 nm) and 590 nm (excitation at 500 nm).

In yet another preferred aspect of the present invention there is provided said Fluorescent carbon nanoparticles (FCNs) comprising said carbon matrix obtained of selective carbohydrate precursor with solvent stabilizer and/or small molecules heteroatom dopant.

In another preferred aspect of the present invention there is provided said Fluorescent carbon nanoparticles wherein said carbon matrix is obtained of selective carbohydrate precursor molecules preferably selected from glucose, glucosamine, dextran, cellulose and said solvents preferably selected from octadecene, octadecene-fatty amine, octadecene-fatty acid, ethylene glycol, amino acids adapted to act as a stabilizer, said heteroatom dopant preferably selected from oxygen, nitrogen.

In yet another preferred aspect of the present invention there is provided said Fluorescent carbon nanoparticles comprising said highly fluorescent carbon nanoparticles involving aqueous and non-aqueous solutions of hydrobhobic/hydrophilic fluorescent carbon nanoparticles adapted to be stable in air and under light irradiation both in solution and in solid form.

In another preferred aspect of the present invention there is provided said Fluorescent carbon nanoparticles comprising heteroatom doped carbon nanoparticles with a preferable narrow size distribution in the range of from 1-5 nm.

In another aspect of the present invention there is provided a method of synthesis of said FCNs comprising the steps of (a) providing carbohydrate molecules;

(b) carbonizing/degrading said carbohydrate; and

(c) controlling the carbon growth conditions/rate of carbonization to obtain said highly fluorescent nanoparticles of desired size and quantum yield by controlling nucleation-growth kinetics involving selectively anyone or more of particle forming precursors, reaction temperature and reaction time.

In another preferred aspect of the present invention there is provided said method of synthesis of FCNs comprising

(a) providing selective carbohydrate molecules for carbonization/degradation with or without involving a selective solvent;
(b) carbonizing/degrading said carbohydrate by heat/boiling or by using concentrated acids with or without the presence of small molecules adapted to passivate the carbon particle surface and/or induce doping of other atoms on the growing carbon particle surface;
(c) controlling the growth conditions/rate of carbonization in the presence or absence of said selective solvents, heating temperature, solution pH and reaction time to yield said FCNs of particle size less than 10 nm in milligram to gram scale in high synthesis yield (>80%) and high quantum yield (>5%).

In yet another preferred aspect of the present invention there is provided said method of synthesis of FCNs wherein said selective carbohydrate molecules for degradation are selected from glucose, glucosamine, dextran and cellulose;

said small molecules are selected from amino acids, DNA bases, degraded products of carbohydrates;

said controlling the growth conditions/rate of carbonization involve controlling the reaction temperature in the range of 50-300° C.; controlling the reaction time from, 1 minute to >10 hours controlling the solution pH from 1 to 12 in the presence of said solvents selected from octadecene, octadecene-fatty amine, octadecene-fatty acid, ethylene glycol or mixtures thereof.

In another preferred aspect of the present invention there is provided said method for the synthesis of FCNs wherein controlling the growth conditions/rate of carbonization comprises the steps of

(a) boiling aqueous carbohydrate solution in presence of said small molecules in different solution pH of 1 to 12 for controlling the carbon particle size/carbonization rate;
(b) injecting additional concentrated carbohydrate solution into a boiling aqueous solution;
(c) terminating the reaction after a time of <1 minute to >1 hour by sudden cooling of reaction flask.

In yet another preferred aspect of the present invention there is provided said method for the synthesis of FCNs comprising the steps of

(a) mixing the aqueous solution of carbohydrate with concentrated sulphuric acid;
(b) carbonizing/degrading said carbohydrate either in room temperature or by mild heating for about 15 minutes to about 2 hours;
(c) providing multiple injections of carbohydrate during the growth stage to control the rate of carbonization to thereby obtain said FCNs.

In another preferred aspect of the present invention there is provided said method for the synthesis of FCNs comprising the steps of

(a) mixing the aqueous solution of carbohydrate with concentrated phosphoric acid;
(b) carbonizing/degrading said carbohydrate by heating to for 80-90° C. for about 1 minute to 5 hours;
(c) neutralizing with sodium hydroxide to obtain highly yellow fluorescent FCNs with emission maxima between 530-590 nm in higher quantum yields.

In yet another preferred aspect of the present invention there is provided said method for the synthesis of FCNs wherein said controlling of the nucleation-growth kinetics is done such as to provide for FCNs obtained in high synthetic yields of >80% and high fluorescence quantum yield between 5-15% and has a size of 1-5 nm with tunable emission colours such as blue, green, yellow, red.

In another aspect of the present invention there is provided Functional FCNs comprising carbon matrix based nanoparticles of upto 10 nm in size with variably tunable emission colours and being highly fluorescent with quantum yield of >5%, said nanoparticles coated with suitable polymer having functional groups.

In another preferred aspect of the present invention there is provided said Functional FCNs comprising hydrophobic FCNs coated with amphiphilic polymer exposed with suitable functional groups such as amine/carboxylate functional groups providing a polymer coated FCN suitable for further functionalization.

In yet another preferred aspect of the present invention there is provided said Functional FCNs comprising intrinsically fluorescent carbon nanoparticles colloidally stable in a medium with pH range of 4 and 10 without any loss of fluorescence activity.

In another preferred aspect of the present invention there is provided said Functional FCNs which is soluble and non-toxic adapted for end use/application as labels in various cellular, sub-cellular, in-vivo and in-vitro imaging and detection techniques.

In another aspect of the present invention there is provided a method for the synthesis of said functional FCNs comprising the steps of

(a) providing the carbon matrix based nanoparticles of upto 10 nm in size with variably tunable emission colours and being highly fluorescent with quantum yield of >5%; and
(b) functionalizing the said nanoparticles with selective polymer coating based functional groups to thereby obtain the functionalized FCNs with retained desired high fluorescence characteristics.

In another preferred aspect of the present invention there is provided said method for the synthesis of functional FCNs comprising:

(a) providing hydrophobic FCNs coated with a fatty amine shell;
(b) coating said hydrophobic FCNs with amphiphilic polymer involving exposed hydrophilic groups selected from anhydrides with the hydrocarbon long chains of said amphiphilic polymers anchored with the fatty amine shell of FCN;
(c) reacting said anhydride groups of polymer with controlled amount of PEG-diamine to thereby obtain functional FCNs functionalized with amine/carboxylate functional groups with retained fluorescence.

In another aspect of the present invention there is provided affinity molecule conjugated functional FCNs comprising polymer coated functional FCNs with carbon matrix based nanoparticles of upto 10 nm in size with variably tunable emission colours and being highly fluorescent with quantum yield of >5% conjugated to different affinity molecules that are soluble, non-toxic possesses good colloidal stability in a medium with pH range between 4 and 10 without any loss of fluorescence property to find its end use/application as labels in various cellular, sub-cellular, in-vivo and in-vitro imaging and detection techniques.

In another preferred aspect of the present invention there is provided said affinity molecule conjugated functional FCNs comprising polymer coated FCNs with suitable exposed functional groups selected from amine/carboxylate functional groups conjugated to the said different affinity molecules.

In yet another preferred aspect of the present invention there is provided said affinity molecule conjugated FCNs wherein said affinity molecules are selected from oleylamine, folic acid, glucose, peptide, antibody.

In another aspect of the present invention there is provided said method for the synthesis of affinity molecule conjugated functional FCNs comprising the steps of conjugating said affinity molecules with polymer coated functionalized FCNs with exposed amine/carboxylate functional groups to thereby obtain said affinity molecule conjugated FCNs with retained fluorescence.

In another aspect of the present invention there is provided a method of delivering affinity molecules inside a cell and/or cell nucleus comprising the steps of (a) administering pre-determined amount of intrinsically fluorescent carbon nanoparticles conjugated to said affinity molecules optionally, along with acceptable additives;

(b) allowing the nanoparticles to penetrate into the cells for about 1-2 hours and get labelled;
(c) imaging the cells under conventional fluorescence microscope thereby favouring cellular, sub-cellular and in-vivo imaging applications.

In another embodiment of the present invention there is provided a method of synthesis for the said functional, soluble and stable fluorescent carbon nanoparticles from hydrophobic/hydrophilic FCNs of the present invention following a conventional coating approach for hydrophobic FCNs to transform them into functionalized FCN with retained fluorescence.

In yet another preferred embodiment of the abovesaid method of the present invention for the synthesis of the functional FCNs, the said Hydrophobic FCNs are coated with amphiphilic polymer with exposed hydrophilic groups suitably functionalized with amine/carboxylate functional groups to generate a polymer coated FCN suitable for further functionalization.

In still another embodiment of the present invention the said polymer coated FCN with amine/carboxylate functional groups is then suitably functionalized with different affinity molecules (such as oleylamine, glucose, peptide, antibody) via different conjugation chemistry.

In yet another embodiment of the present invention, there is provided a water soluble and functional fluorescent carbon nanoparticles (FCNs) possessing good colloidal stability in a medium with pH range between 4 and 10 without any loss of fluorescence property.

In still another embodiment of the present invention, the fluorescent carbon nanoparticles (FCNs) of the present invention are non-toxic and finds its end use/application as labels in various cellular, sub-cellular, in-vivo and in-vitro imaging and detection techniques.

### BRIEF DESCRIPTION OF THE DRAWINGS

FIG. 1 illustrates different controlled carbonization approach of carbohydrate in producing fluorescent carbon nanoparticles (FCN).

FIG. 2 illustrates Fluorescence spectra of FCN having different emission color.

FIG. 3 illustrates TEM image of hydrophobic green emitting FCN with 1.5 nm average sizes.

FIG. 4(a) illustrates FTIR spectra of carbohydrate-octadecylamine reaction mixture at different reaction times; initial stage (within 1-2 minutes), middle stage (5 minutes) and final stages (10 minutes). Data shows aldehyde formation at the intermediate stage with the appearance of strong peaks between 1700-1750 cm⁻¹ and FIG. 4(b) FTIR spectra of purified FCN showing mainly the presence of octadecylamine.

FIGS. 5(a) and 5(b) illustrates Results of fluorescence stability test of FCN, As for FIG. 5(a), dried drop of FCN solution in glass slide was irradiated with light and imaged using Olympus IX 81 microscope attached with a digital camera. Images show little change with continuous light irradiation. FIG. 5(b) shows that polymer coated FCN dissolved in buffer solution of different solution pH, showing that emission intensity does not change appreciably with pH.

FIG. 6 illustrates Polymer coating strategy for hydrophobic FCN to convert into hydrophilic FCN.

FIG. 7 illustrates Digital image of hydrophobic green emitting FCN solution in toluene (left), the same solution under hand held UV lamp (middle) and polymer coated hydrophilic FCN in presence of UV light (right).

FIG. 8 illustrates Bright field (left panel) and fluorescence (right panel) image of oleyl functionalized FCN labeled COS-7 cells.

FIG. 9 illustrates confocal fluorescence microscopic image of folic acid functionalized FCN labeled HeLa cells. Blue color represents the labeled FCN.

### DETAILED DESCRIPTION OF THE EXEMPLARY EMBODIMENTS

As described hereinbefore the present invention provides for highly fluorescent carbon nanoparticles involving aqueous and non-aqueous solutions of hydrophobic/hydrophilic fluorescent carbon nanoparticle (FCN) with tunable emission colours such as blue, green, yellow, red and of particle size 1-10 nm preferably 1-5 nm that is also stable in solid form and a chemical synthesis method for obtaining the same. The FCNs generated in the process has a size of 1-5 nm, involves a high synthesis yield (>80%), high fluorescence quantum yield (between 5-15%). The method of the present invention involves some specific carbohydrate molecules as carbon sources wherein the FCNs are produced via carbonization of those carbohydrate molecules using controlled growth conditions. The said method generates FCN of different emission colors simply by tuning the growth condition or by changing the carbohydrate precursors.

The successful synthesis of high quality FCNs in the present invention is due to the selection of specific carbohydrate molecules that provide doping of other atoms (such as oxygen, nitrogen) on carbon particle and their carbonization in a controlled manner to produce highly fluorescent carbon nanoparticles with quantum yield of >5% and with smaller size (in the 1-5 nm range) with narrow size distribution.

The high quality FCN of the present invention and its successful synthetic procedure rests on the selective finding of some specific carbohydrate molecules that encompasses the provision of doping by other atoms (such as oxygen, nitrogen) on carbon particle and their carbonization in a controlled manner to produce carbon particles (FCNs) of smaller size of 1-5 nm range and with narrow size distribution.

Thus key features of the present method are the formation of smaller size carbon particle in the controlled growth condition and doping of other elements in such small size carbon particle.

The following examples are given by the way of illustration of the present invention and therefore should not be construed to limit the scope of the present invention.

### Example 1

### Preparation Method of the Fluorescent Carbon Nanoparticles

FCN is produced by controlled carbonization of carbohydrate molecule. Typically, a carbohydrate molecule is dissolved in a solvent and carbonization is performed either by heating at high temperature or using concentrated sulphuric acid. Rate of carbonization is controlled by heating temperature, solvent composition, solution pH and reaction time. Different approaches are used for controlled carbonization. In one approach solid carbohydrate molecule is mixed with long chain fatty amine and heated at high temperature. Although this approach is similar to the size controlled synthesis of metal and metal oxide nanoparticle by thermal degradation of metallic precursors, the carbohydrate degradation has not been studied in this growth condition. Advantage of this approach is that particle size can be controlled by controlling the nucleation-growth kinetics using different particle forming precursors, changing reaction temperature and reaction time. In the present case FCN of different emission color and of higher quantum yield has been obtained using different carbohydrate molecules (such as glucose, glucosamine, dextran and cellulose), controlling the reaction temperature, changing the reaction time and using different solvents (such as octadecene, octadecene-fatty amine, octadecene-fatty acid, ethylene glycol). During heating processes the solid carbohydrate is dissolved in liquid fatty amine, followed by the appearance of yellow-brown color. Hydrophobic FCN are formed in this approach and formation of this product is monitored by observing the emission color in different reaction time.

In another approach carbonization was performed by boiling aqueous carbohydrate solution in presence of other small molecules (such as amino acids) and in different solution pH. In order to have a better control of the carbon particle size, the concentrated carbohydrate solution is injected into a boiling aqueous solution and reaction has been stopped at a desired time by sudden cooling of reaction flask. Different small molecules are added in the growth condition so that they can passivate the carbon particle surface and/or induce doping of other atoms on the growing carbon particle surface. Different solution pH helps to control the carbonization rate.

### Example 2

### Method of Preparing FCN

Aqueous solution of carbohydrate (such as glucose, glucosamine, dextran and cellulose) is mixed with concentrated sulfuric acid and carbonization has been performed either in room temperature or by mild heating for 15 minutes to 2 hours. Although the sulphuric acid based method is used earlier, in the present case the carbonization rate is controlled by using different carbohydrate molecules, reaction temperature and multiple injections of carbohydrate precursors during the growth stage. These modifications provide FCN with tunable emission colors with enhanced quantum yield.

In another approach yellow fluorescent carbon nanoparticles has been synthesized by carbonization of carbohydrate in concentrated phosphoric acid. Typically, carbohydrate is dissolved in concentrated phosphoric acid and heated to 80-90° C. for 1 minute to 5 hours and then reaction mixture is neutralized with sodium hydroxide. The resultant solution is highly yellow fluorescent with emission maxima between 530-560 nm. In comparison to the sulphuric acid based method this approach produces yellow and red emitting FCN with better quantum yield.

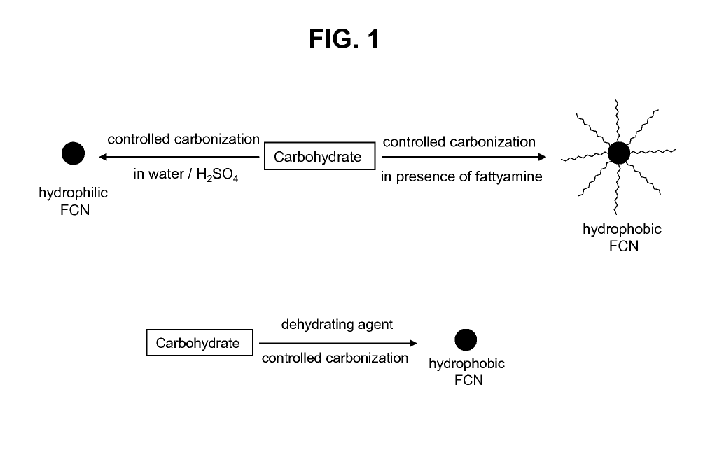

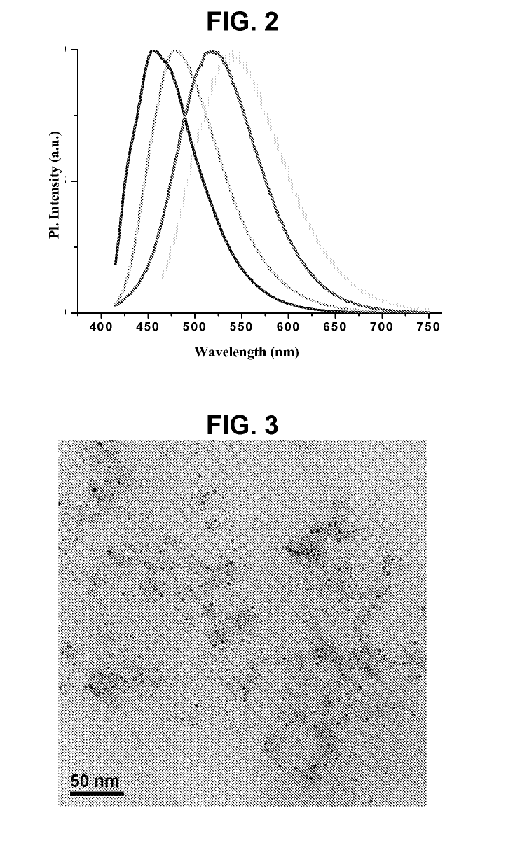

The carbohydrate carbonization approach described in the FIG. 1 can produce FCN of different emission color such as blue, green and yellow depending on the nature of carbohydrate precursor and carbonization condition. FIG. 2 shows the emission spectra of FCN with four different emission colors. The emission peak is 455 nm (excitation at 350 nm), 480 nm (excitation at 400 nm), 520 nm (excitation at 400 nm), and 540 nm (excitation at 450 nm). The fluorescence quantum yield (QY) measured for these samples, ranges between 5-15%, using fluorescein/quinine sulfate as references.

### Example 3

### Characterization of FCN

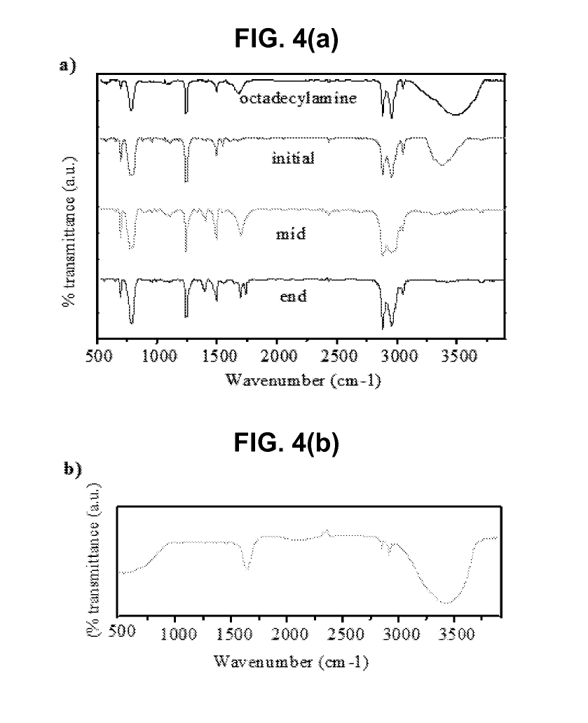

FCN has been characterized by different methods such as elemental analysis, Fourier transform infrared spectroscopy (FTIR), proton NMR, TGA, transmission electron microscopy (TEM) and Raman spectroscopy. Elemental analysis shows the presence of elemental carbon above 75% along with hydrogen, nitrogen and oxygen. However, the elemental composition varies depending on the nature of precursor carbohydrate used and presence of surface adsorbed molecules. TEM study shows that smaller particles of 1-3 nm diameters are formed during the carbonization processes. FIG. 3 shows the TEM image of green emitting hydrophobic FCN with average diameter of 1.5 nm, which is prepared using long chain fatty amine as solvent and stabilizer. FTIR study has been performed to study the reaction mechanism as well as to understand the composition of surface ligands. FIGS. 4(a) and 4(b) show the FTIR spectra of the reaction mixture at different stages during the formation of FCN from carbohydrate in octadecylamine medium. It shows the formation of intermediate aldehyde during the carbohydrate carbonization with the signature of peaks in the range of 1700-1750 cm⁻¹. Purified hydrophobic FCN shows strong peaks due to octadecylamine, suggesting that FCN has octadecylamine as surface ligands.

The small size of the carbon particle (<5 nm) and presence of other doped elements such as nitrogen/oxygen and their relative ratio in the carbon matrix dictate the quantum yield of the carbon particle.

Thus key features of the present advancement reside in the formation of smaller size carbon nanoparticles under said controlled growth condition and doping of other elements in such small size carbon particle.

### Example 4

### Biomedical Applications of the Prepared FCN

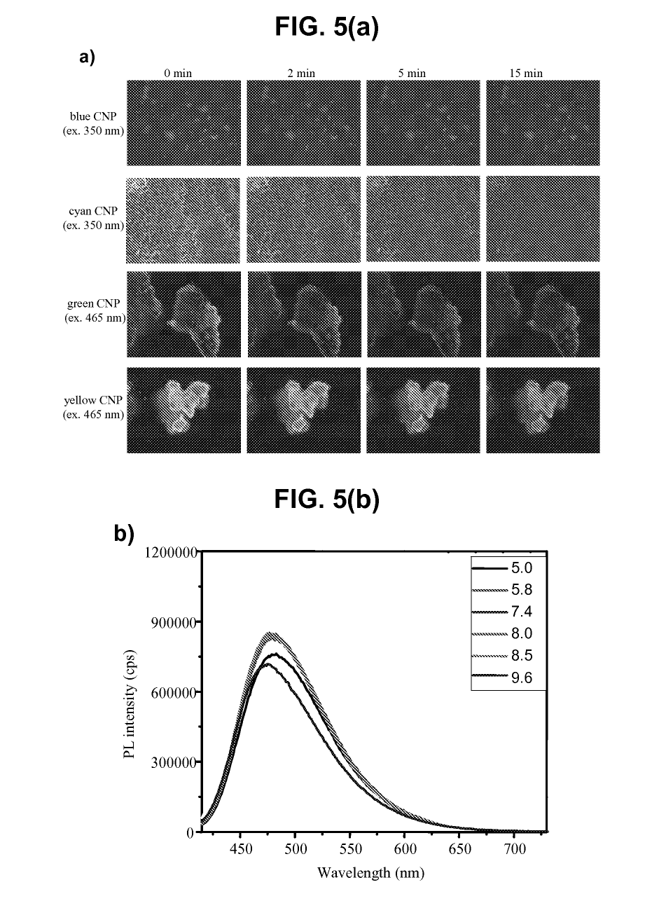

In order to understand the application potential of FCN, we have synthesized different functional FCN, investigated their fluorescence stability and used them as fluorescent cellular label. FIGS. 5(a) and 5(b) show the fluorescence property of green fluorescent FCN in different pH and under continuous UV exposure time. Result shows that fluorescence of FCN is stable in different buffer solutions and they do not photo bleach under UV exposure. These results ensure that FCN can be used as imaging probe under adverse cellular environment and for continuous monitoring of target molecule, similar to quantum dot.

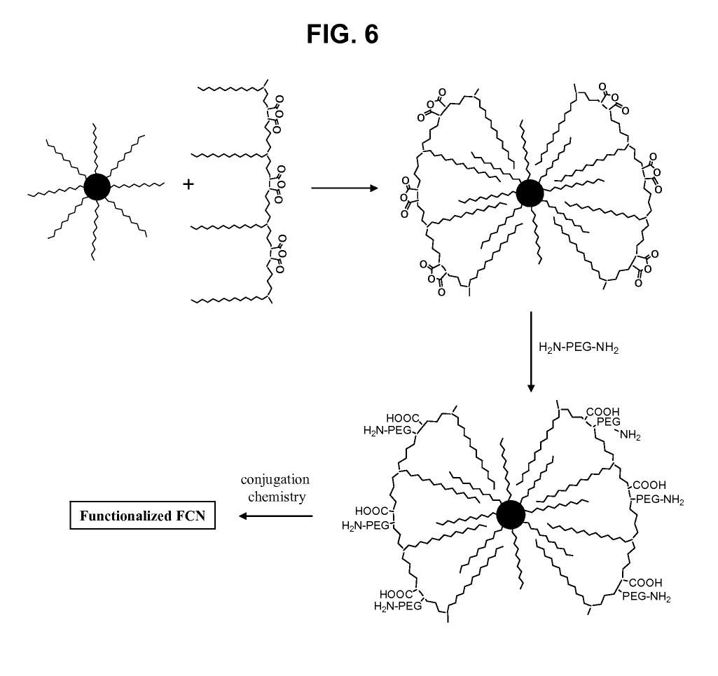

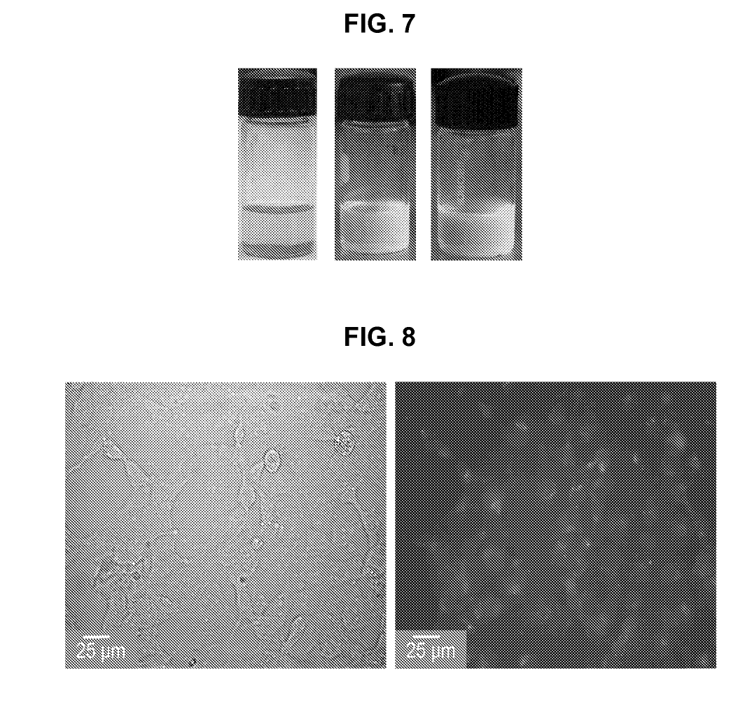

Various functionalized FCN are required for different bio-labeling applications and it is necessary to test if the as synthesized hydrophobic/hydrophilic FCNs can be converted into functional FCN with retained fluorescence. We have applied a well established coating approach for hydrophobic FCN and successfully transformed them into functionalized FCN with retained fluorescence. (FIG. 6, FIG. 7) Hydrophobic FCN is coated with amphiphilic polymer where hydrocarbon long chains of polymers are anchored with the fatty amine shell of FCN and hydrophilic anhydride groups of polymer are reacted with controlled amount of PEG-diamine. As a result hydrophobic FCN is transformed into polymer coated hydrophilic FCN with terminal primary amine and carboxylate functional groups. The polymer coated FCN is then functionalized with different affinity molecules (such as oleylamine, folic acid, glucose, peptide, antibody) via different conjugation chemistry using the primary amine/carboxylate functional groups. We found that our FCN can be transformed into functional FCN using conventional coating and conjugation chemistry and other functional FCN can also be prepared. Thus our FCN can be used to prepare variety of functional FCN for different biomedical applications.

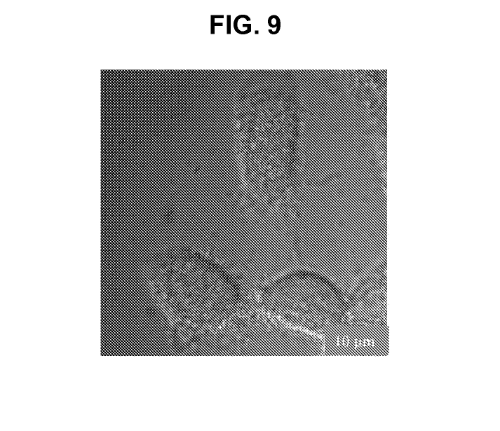

In order to test that the functional FCN can be used as cellular probe, investigations regarding the performance of oleylamine functionalized FCN has been made. Oleyl/folic acid functionalized FCN is mixed with cell culture medium having cells attached on the cultured plates. We found that cells get labeled within 1-2 hours and labeled cells can be imaged with conventional fluorescence microscope. (FIG. 8 and FIG. 9) Other functional FCNs were synthesized and used for cellular, sub-cellular and in-vivo imaging applications.

It is thus possible by way of the present invention to provide for highly fluorescent carbon nanoparticles (FCNs) involving aqueous and non-aqueous solutions of hydrophobic/hydrophilic FCNs with tunable emission colours of particle size between 1-10 nm that is also stable in solid form. The present invention further relates to the method of synthesis of the said FCNs involving carbohydrate degradation under controlled growth conditions to yield said FCNs in milligram to gram scale in high quantum yield (>5%) and in high synthetic yield (>80%). The present invention is further directed to highly fluorescent carbon nanoparticle solution doped with heteroatom (such as oxygen, nitrogen) and its method of synthesis involving controlled carbonization to yield said doped carbon nanoparticles of even smaller size ranging from 1-5 nm with narrow size distribution. Advantageously, the present invention also provides for functionalized FCNs that are non-toxic, functional, soluble and stable with retained fluorescence and its method of synthesis to finds its end use in biomedics, imaging applications, and detection techniques.

We claim:

1. Fluorescent carbon nanoparticles (FCNs) comprising carbon matrix based nanoparticles of upto 10 nm in size with variably tunable emission colours and being highly fluorescent with quantum yield of >5%.

2. Fluorescent carbon nanoparticles (FCNs) as claimed in claim 1 having high fluorescent quantum yield in the range of 5-15% and tunability of emission colours of blue, green, yellow, red with emission peaks at 455 nm (excitation at 350 nm), 480 nm (excitation at 400 nm), 520 nm (excitation at 400 nm), 540 nm (excitation at 450 nm) and 590 nm (excitation at 500 nm).

3. Fluorescent carbon nanoparticles (FCNs) as claimed in claim 1 comprising said carbon matrix obtained of selective carbohydrate precursor with solvent stabilizer and/or small molecules heteroatom dopant.

4. Fluorescent carbon nanoparticles as claimed in claim 1 wherein said carbon matrix is obtained of selective carbohydrate precursor molecules preferably selected from glucose, glucosamine, dextran, cellulose and said solvents preferably selected from octadecene, octadecene-fatty amine, octadecene-fatty acid, ethylene glycol, amino acids adapted to act as a stabilizer, said heteroatom dopant preferably selected from oxygen, nitrogen.

5. Fluorescent carbon nanoparticles as claimed in claim 1 comprising said highly fluorescent carbon nanoparticles involving aqueous and non-aqueous solutions of hydrophobic/hydrophilic fluorescent carbon nanoparticles adapted to be stable in air and under light irradiation both in solution and in solid form.

6. Fluorescent carbon nanoparticles as claimed in claim 3 comprising heteroatom doped carbon nanoparticles with a preferable narrow size distribution in the range of from 1-5 nm.

7. A method of synthesis of FCNs as claimed in claim 1 comprising the steps of
(a) providing carbohydrate molecules;
(b) carbonizing/degrading said carbohydrate; and
(c) controlling the carbon growth conditions/rate of carbonization to obtain said highly fluorescent nanoparticles of desired size and quantum yield by controlling nucleation-growth kinetics involving selectively anyone or more of particle forming precursors, reaction temperature and reaction time.

8. A method of synthesis of FCNs as claimed in claim 7 comprising
(a) providing selective carbohydrate molecules for carbonization/degradation with or without involving a selective solvent;
(b) carbonizing/degrading said carbohydrate by heat/boiling or by using concentrated acids with or without the presence of small molecules adapted to passivate the carbon particle surface and/or induce doping of other atoms on the growing carbon particle surface;
(c) controlling the growth conditions/rate of carbonization in the presence or absence of said selective solvents, heating temperature, solution pH and reaction time to yield said FCNs of particle size less than 10 nm in milligram to gram scale in high synthesis yield (>80%) and high quantum yield (>5%).

9. A method of synthesis of FCNs as claimed in claim 8 wherein said selective carbohydrate molecules for degradation are selected from glucose, glucosamine, dextran and cellulose;
said small molecules are selected from amino acids, DNA bases, degraded products of carbohydrates;
said controlling the growth conditions/rate of carbonization involve controlling the reaction temperature in the range of 50-300° C.; controlling the reaction time from <1 minute to >10 hours, controlling the solution pH from 1 to 12 in the presence of said solvents selected from octadecene, octadecene-fatty amine, octadecene-fatty acid, ethylene glycol or mixtures thereof.

10. A method for the synthesis of FCNs as claimed in claim 7 wherein controlling the growth conditions/rate of carbonization comprises the steps of
(a) boiling aqueous carbohydrate solution in presence of said small molecules in different solution pH of 1 to 12 for controlling the carbon particle size/carbonization rate;
(b) injecting additional concentrated carbohydrate solution into a boiling aqueous solution;
(c) terminating the reaction after a time of <1 minute to >1 hour by sudden cooling of reaction flask.

11. A method for the synthesis of FCNs as claimed in claim 7 comprising the steps of
(a) mixing the aqueous solution of carbohydrate with concentrated sulphuric acid;
(b) carbonizing/degrading said carbohydrate either in room temperature or by mild heating for about 15 minutes to about 2 hours;
(c) providing multiple injections of carbohydrate during the growth stage to control the rate of carbonization to thereby obtain said FCNs.

12. A method for the synthesis of FCNs as claimed in claim 7 comprising the steps of
(a) mixing the aqueous solution of carbohydrate with concentrated phosphoric acid;
(b) carbonizing/degrading said carbohydrate by heating to for 80-90° C. for about 1 hour to 5 hours;
(c) neutralizing with sodium hydroxide to obtain highly yellow fluorescent FCNs with emission maxima between 530-590 nm in higher quantum yields.

13. A method for the synthesis of FCNs as claimed in claim 7 wherein said controlling of the nucleation-growth kinetics is done such as to provide for FCNs obtained in high synthetic yields of >80% and high fluorescence quantum yield between 5-15% and has a size of 1-5 nm with tunable emission colours such as blue, green, yellow, red.

14. Functional FCNs comprising carbon matrix based nanoparticles of upto 10 nm in size with variably tunable emission colours and being highly fluorescent with quantum yield of >5%, said nanoparticles coated with suitable polymer with functional groups.

15. Functional FCNs as claimed in claim 14 comprising hydrophobic FCNs coated with amphiphilic polymer exposed with suitable functional groups such as amine/carboxylate functional groups providing a polymer coated FCN suitable for further functionalization.

16. Functional FCNs as claimed in claim 14 comprising intrinsically fluorescent carbon nanoparticles colloidally stable in a medium with pH range of 4 and 10 without any loss of fluorescence activity.

17. Functional FCNs as claimed in claim 14 which is soluble and non-toxic adapted for end use/application as labels in various cellular, sub-cellular, in-vivo and in-vitro imaging and detection techniques.

18. A method for the synthesis of functional FCNs as claimed in claim 14 comprising the steps of
(a) providing the carbon matrix based nanoparticles of upto 10 nm in size with variably tunable emission colours and being highly fluorescent with quantum yield of >5%; and
(b) functionalizing the said nanoparticles with selective polymer coating based functional groups to thereby obtain the functionalized FCNs with retained desired high fluorescence characteristics.

19. A method for the synthesis of functional FCNs as claimed in claim 18 comprising:
(a) providing hydrophobic FCNs coated with a fatty amine shell;
(b) coating said hydrophobic FCNs with amphiphilic polymer involving exposed hydrophilic groups selected from anhydrides with the hydrocarbon long chains of said amphiphilic polymers anchored with the fatty amine shell of FCN;
(c) reacting said anhydride groups of polymer with controlled amount of PEG-diamine to thereby obtain functional FCNs functionalized with amine/carboxylate functional groups with retained fluorescence.

20. Affinity molecule conjugated functional FCNs comprising polymer coated functional FCNs with carbon matrix based nanoparticles of upto 10 nm in size with variably tunable emission colours and being highly fluorescent with quantum yield of >5% conjugated to different affinity molecules that are soluble, non-toxic possesses good colloidal stability in a medium with pH range between 4 and 10 without any loss of fluorescence property to find its end use/application as labels in various cellular, sub-cellular, in-vivo and in-vitro imaging and detection techniques.

21. Affinity molecule conjugated functional FCNs as claimed in claim 20 comprising polymer coated FCNs with suitable exposed functional groups selected from amine/carboxylate functional groups conjugated to the said different affinity molecules.

22. Affinity molecule conjugated FCNs as claimed in claim 20 wherein said affinity molecules are selected from oleylamine, folic acid, glucose, peptide, antibody.

23. A method for the synthesis of affinity molecule conjugated functional FCNs as claimed in claim 20 comprising the steps of conjugating said affinity molecules with polymer coated functionalized FCNs with exposed amine/carboxylate functional groups to thereby obtain said affinity molecule conjugated FCNs with retained fluorescence.

24. A method of delivering affinity molecules inside a cell and/or cell nucleus comprising the steps of (a) administering pre-determined amount of intrinsically fluorescent carbon nanoparticles conjugated to affinity molecules as claimed in claim 20 optionally, along with acceptable additives;
(b) allowing the nanoparticles to penetrate into the cells for about 1-2 hours and get labelled;
(c) imaging the cells under conventional fluorescence microscope thereby favouring cellular, sub-cellular and in-vivo imaging applications.
